(19) United States
(12) Patent Application Publication
(10) Pub. No.: US 2014/0330359 A1
(43) Pub. Date: Nov. 6, 2014

(54) METHODS AND APPARATUS FOR STENT DELIVERY AND DEPLOYMENT

(71) Applicant: Amaranth Medical Pte., Singapore (SG)

(72) Inventors: Kamal RAMZIPOOR, Fremont, CA (US); Chang Y. LEE, Redwood City, CA (US); Edward A. ESTRADA, Menlo Park, CA (US)

(73) Assignee: AMARANTH MEDICAL PTE., Singapore (SG)

(21) Appl. No.: 14/252,488

(22) Filed: Apr. 14, 2014

Related U.S. Application Data
(60) Provisional application No. 61/819,337, filed on May 3, 2013.

(51) Int. Cl. A61F 2/958

(52) U.S. Cl. CPC A61F 2/958
USPC 623/1.11

(57) ABSTRACT

For scaffolds delivered and deployed intravascularly, one or several configurations may be employed for creating a smoother transition from balloon-to-scaffold. For instance, one or more spacers may be positioned in proximity to the scaffold. Alternatively, portions of the inflatable balloon may be configured to provide a smooth transition. In another alternative, the stent edges may be shaped to provide a smooth transition. In yet other variations, any number of different combinations of such features may be employed.

### CROSS-REFERENCE TO RELATED APPLICATIONS

This application claims the benefit of priority to U.S. Prov. App. 61/819,337 filed May 3, 2013, which is incorporated herein by reference in its entirety.

### FIELD OF THE INVENTION

The present invention relates generally to stent or scaffold delivery and deployment methods and apparatus. More particularly, the present invention relates to methods and apparatus for providing a smooth transition between a crimped stent and inflation balloon for facilitating intravascular delivery and deployment of the stent or scaffold.

### BACKGROUND OF THE INVENTION

When a scaffold or stent 12 is crimped upon an inflation balloon 14 for delivery via a catheter 10 within the vasculature, the edges 16 of the scaffold 12 may protrude radially at the balloon-scaffold transition, as shown in the side view of FIG. 1A, and prevent smooth intravascular navigation to the target site.

Accordingly, it is desirable to provide for a smooth transition region between the balloon or catheter and the scaffold or stent crimped or otherwise secured upon the balloon.

### SUMMARY OF THE INVENTION

For scaffolds delivered and deployed intravascularly, one or several configurations may be employed for creating a smoother transition from balloon-to-scaffold. In one variation, a scaffold delivery apparatus may generally comprise a catheter having an inflatable balloon thereupon, an expandable scaffold secured upon the inflatable balloon, and a distal spacer positioned beneath or within the inflatable balloon at a location distal to the scaffold, wherein the distal spacer is tapered to widen from a distal end to a proximal end adjacent to the scaffold.

In another variation, the scaffold delivery apparatus may generally comprise an inflatable balloon positioned upon a catheter, and an expandable scaffold secured upon the inflatable balloon, wherein a distal portion of the inflatable balloon defines a tapered transition adjacent to a distal end of the scaffold and a proximal portion of the inflatable balloon defines a tapered transition adjacent to the proximal end of the scaffold.

In another variation, the scaffold delivery apparatus may generally comprise an inflatable balloon positioned upon a catheter, and an expandable scaffold secured upon the inflatable balloon, wherein the scaffold defines at least a distal tapered edge.

In another variation, the scaffold delivery apparatus may generally comprise a catheter having an inflatable balloon thereupon, an expandable scaffold secured upon the inflatable balloon, a distal spacer positioned beneath or within the inflatable balloon at a location distal to the scaffold, wherein the distal spacer is tapered to widen from a distal end to a proximal end adjacent to the scaffold, and wherein a distal portion of the inflatable balloon defines a tapered transition adjacent to a distal end of the scaffold and a proximal portion of the inflatable balloon defines a tapered transition adjacent to the proximal end of the scaffold.

In another variation, the scaffold delivery apparatus may generally comprise a catheter having an inflatable balloon thereupon, an expandable scaffold secured upon the inflatable balloon, a distal spacer positioned beneath or within the inflatable balloon at a location distal to the scaffold, wherein the distal spacer is tapered to widen from a distal end to a proximal end adjacent to the scaffold, and wherein the scaffold defines at least a distal tapered edge.

In another variation, the scaffold delivery apparatus may generally comprise a catheter having an inflatable balloon thereupon, an expandable scaffold secured upon the inflatable balloon, wherein a distal portion of the inflatable balloon defines a tapered transition adjacent to a distal end of the scaffold and a proximal portion of the inflatable balloon defines a tapered transition adjacent to the proximal end of the scaffold, and wherein the scaffold defines at least a distal tapered edge.

In another variation, the scaffold delivery apparatus may generally comprise a catheter having an inflatable balloon thereupon, an expandable scaffold secured upon the inflatable balloon, a distal spacer positioned beneath or within the inflatable balloon at a location distal to the scaffold, wherein the distal spacer is tapered to widen from a distal end to a proximal end adjacent to the scaffold, wherein a distal portion of the inflatable balloon defines a tapered transition adjacent to a distal end of the scaffold and a proximal portion of the inflatable balloon defines a tapered transition adjacent to the proximal end of the scaffold, and wherein the scaffold defines at least a distal tapered edge.

In another variation, the scaffold delivery method may generally comprise providing a catheter having an inflatable balloon thereupon, an expandable scaffold secured upon the inflatable balloon, and a distal spacer positioned beneath or within the inflatable balloon at a location distal to the scaffold, wherein the distal spacer is tapered to widen from a distal end to a proximal end adjacent to the scaffold, and inflating the balloon such that the scaffold is expanded radially beyond the distal spacer.

### BRIEF DESCRIPTION OF THE DRAWINGS

FIG. 1A shows a side view of a scaffold crimped upon a balloon illustrating the exposed scaffold edges which may hinder navigation of the assembly through the vasculature.

FIGS. 1B and 1C show side views of one variation of an assembly having tapered spacers underneath or within the distal and proximal portions of the inflation balloon which prevent radial exposure of the scaffold edges.

FIGS. 2A and 2B show side views of another variation of an assembly where the distal and proximal portions of the inflation balloon abutting the scaffold may be specially shaped in a tapered manner to prevent radial exposure of the scaffold edges.

FIGS. 3A and 3B show side views of yet another variation of an assembly where the distal and proximal portions of the scaffold may be shaped or tapered to present a smooth transition between the catheter, inflation balloon, and scaffold.

### DETAILED DESCRIPTION OF THE INVENTION

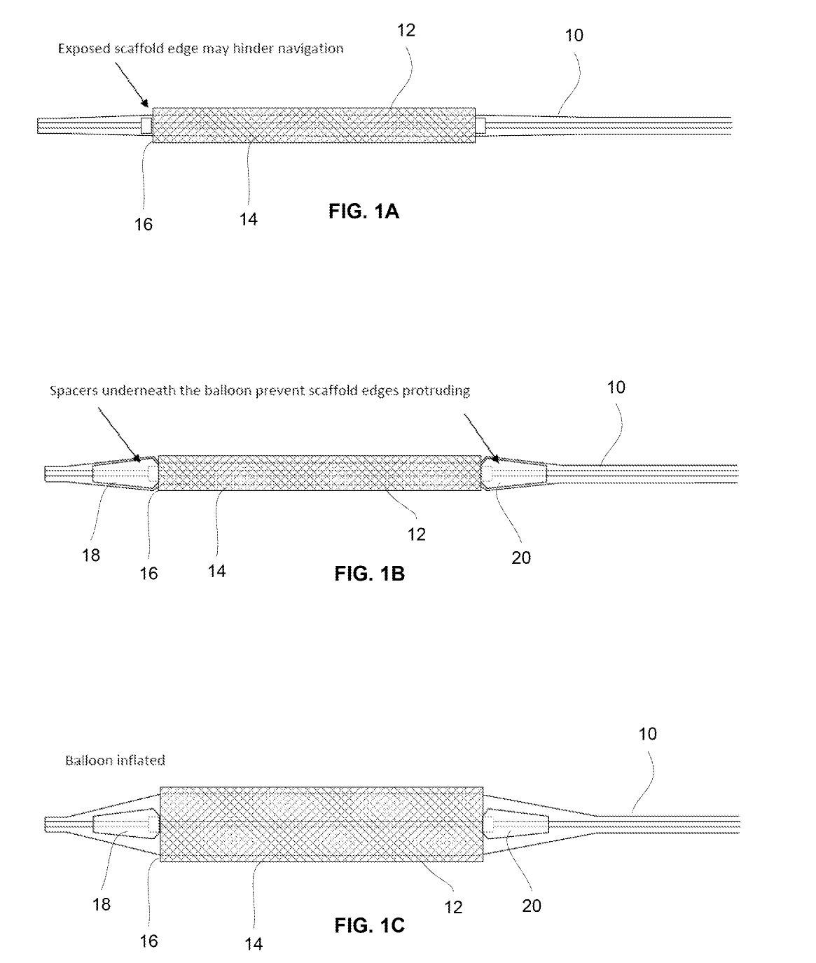

For scaffolds 12 with wall thicknesses greater than 50 μm, it is possible to improve intravascular navigation by creating a smoother transition from balloon-to-scaffold. One method of creating this smooth transition is to place spacers underneath or within the balloon 14 that will increase the diameter of balloon 14 just prior and/or just after the scaffold, as shown in the side view of FIG. 1B. A distal spacer 18 which is tapered to widen radially from its distal end to its proximal end may be positioned distal to the scaffold 12. A similar proximal spacer 20 which is tapered to narrow radially from its distal end to its proximal end may be positioned proximal to the scaffold 12 as well. The widened ends of the spacers 18, 20 may have a diameter which is similar or the same as the diameter of the crimped scaffold 12 secured upon the balloon 14 to prevent the scaffold edges 16 from protruding beyond the spacers 18, 20 during intravascular delivery. Alternatively, depending on the crimped diameter of the scaffold 12 the diameter of the crimped balloon 14 at the spacer regions can range from one wall thickness less than the crimped diameter of the scaffold 12 to two wall thickness greater than crimped diameter of the scaffold 12.

When the scaffold 12 is to be deployed, the inflation balloon 14 may be simply expanded as normally done to expand the scaffold 12 beyond the diameter of the spacers 18, 20, as shown in the side view of FIG. 1C.

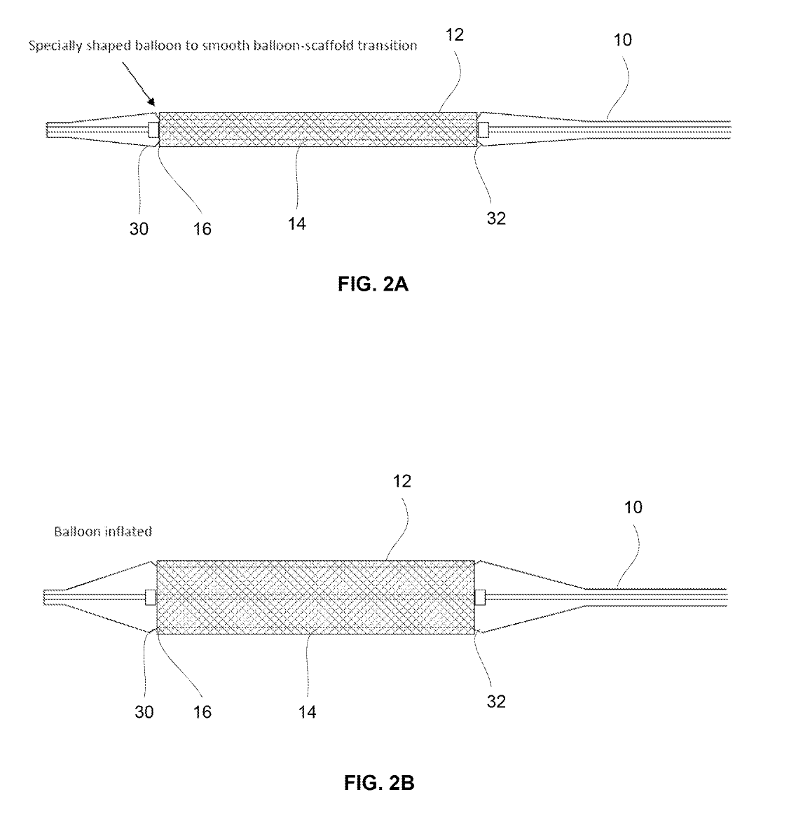

Another variation is illustrated in the side views of FIGS. 2A and 2B which show an inflation balloon 14 which is specially configured to define a step 30, 32 in the balloon 14 to accommodate the diameter change due to scaffold wall thickness. The distal step 30 may be defined by the balloon 14 distal to the scaffold 12 and the proximal step 32 may be defined by the balloon 14 proximal to the scaffold 12 such that the steps 30, 32 abut the scaffold 12 in a tapered manner to present a smooth balloon-scaffold transition. The balloon 14 may be inflated normally for deployment into the body lumen.

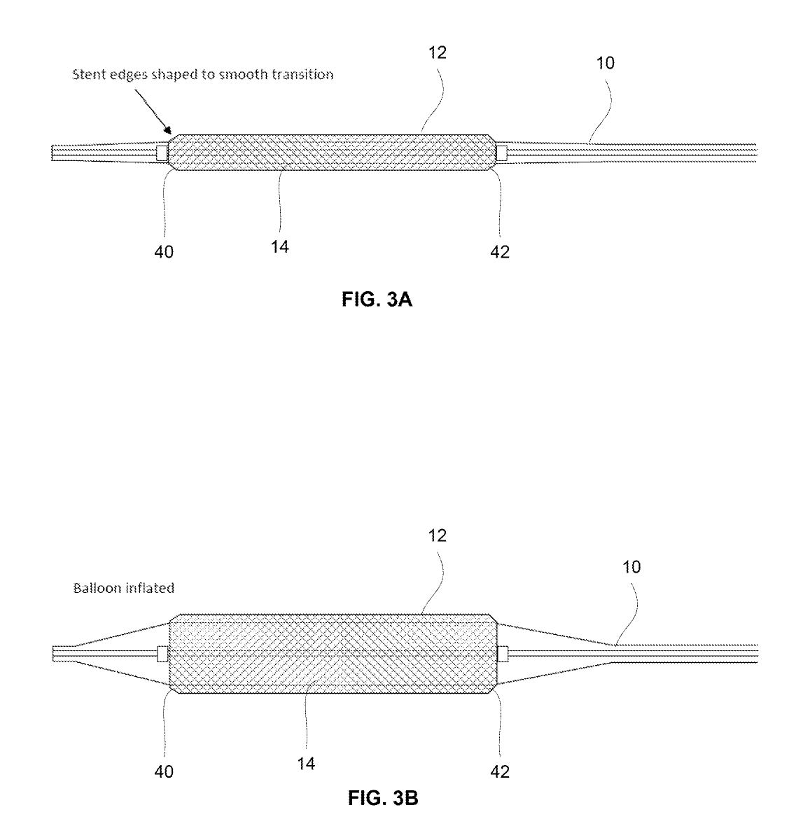

FIGS. 3A and 3B show side views of yet another variation where the distal 40 and proximal edges 42 of the scaffold 12 may be tapered. The transitioning scaffold edges 40. 42 can be created using methods including, e.g., angled laser beam, profile machining, control depth profile machining, using laser beam or other mechanisms, injection molding process, abrasive or material removal process, as well as other material removal/deposit processes. The balloon 14 may be simply inflated as normal for stent deployment, as shown in FIG. 3B.

Lastly, the stent 14 can also be made thinner to smooth the balloon-scaffold transition. The thinned stent 14 as well as each of the transition features described herein may be used in any number of combinations. For example, the spacers 18, 20 may be used in combination with the tapered balloon edges 30, 32 in a single assembly. Alternatively, these features may also be used in combination with the tapered scaffold edges 40, 42 in any variation.

Moreover, the transition features described herein may be used with any number of scaffolds or stent structures and particularly with polymeric substrate and stent assemblies as described in U.S. patent application Ser. Nos. 10/867,617 filed Jun. 15, 2004 (U.S. Pub. 2005/0021131); 13/476,853 filed May 21, 2012 (U.S. Pub. 2012/0232643); 13/476,858 filed May 21, 2012 (U.S. Pub. 2012/0232644); 12/541,095 filed Aug. 13, 2009 (U.S. Pub. 2010/0042202); U.S. Pat. Nos. 8,206,635; 8,206,636; and 8,309,023. Each of these references is incorporated herein by reference in its entirety for any purpose.

Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A scaffold delivery apparatus, comprising:
a catheter having an inflatable balloon thereupon;
an expandable scaffold secured upon the inflatable balloon; and
a distal spacer positioned beneath or within the inflatable balloon at a location distal to the scaffold,
wherein the distal spacer is tapered to widen from a distal end to a proximal end adjacent to the scaffold.

2. The apparatus of claim 1 further comprising a proximal spacer positioned beneath or within the inflatable balloon at a location proximal to the scaffold.

3. The apparatus of claim 2 wherein the proximal spacer is tapered to narrow from a distal end adjacent to the scaffold to a proximal end.

4. The apparatus of claim 1 wherein a widened end of the distal spacer defines a diameter which is similar to a diameter of the scaffold when crimped upon the balloon to prevent scaffold edges from protruding beyond the spacer.

5. The apparatus of claim 4 wherein expansion of the scaffold for deployment expands the scaffold to a second diameter which is larger than the diameter of the spacer.

6. The apparatus of claim 1 wherein a distal portion of the inflatable balloon defines a tapered transition adjacent to a distal end of the scaffold and a proximal portion of the inflatable balloon defines a tapered transition adjacent to the proximal end of the scaffold.

7. The apparatus of claim 1 wherein the scaffold defines at least a distal tapered edge.

8. A scaffold delivery apparatus, comprising:
an inflatable balloon positioned upon a catheter; and
an expandable scaffold secured upon the inflatable balloon,
wherein the scaffold defines at least a distal tapered edge.

9. The apparatus of claim 8 further comprising a distal spacer positioned beneath or within the inflatable balloon at a location distal to the scaffold, wherein the distal spacer is tapered to widen from a distal end to a proximal end adjacent to the scaffold, and

10. The apparatus of claim 8 wherein a distal portion of the inflatable balloon defines a tapered transition adjacent to a distal end of the scaffold and a proximal portion of the inflatable balloon defines a tapered transition adjacent to the proximal end of the scaffold, and wherein the scaffold defines at least a distal tapered edge.

11. The apparatus of claim 8 further comprising a distal spacer positioned beneath or within the inflatable balloon at a location distal to the scaffold,
wherein the distal spacer is tapered to widen from a distal end to a proximal end adjacent to the scaffold,
wherein a distal portion of the inflatable balloon defines a tapered transition adjacent to a distal end of the scaffold and a proximal portion of the inflatable balloon defines a tapered transition adjacent to the proximal end of the scaffold, and
wherein the scaffold defines at least a distal tapered edge.

12. A scaffold delivery apparatus, comprising:
an inflatable balloon positioned upon a catheter; and
an expandable scaffold secured upon the inflatable balloon,
wherein a distal portion of the inflatable balloon defines a tapered transition adjacent to a distal end of the scaffold and a proximal portion of the inflatable balloon defines a tapered transition adjacent to the proximal end of the scaffold.

13. A scaffold delivery apparatus, comprising:
a catheter having an inflatable balloon thereupon;
an expandable scaffold secured upon the inflatable balloon;
a distal spacer positioned beneath or within the inflatable balloon at a location distal to the scaffold,
wherein the distal spacer is tapered to widen from a distal end to a proximal end adjacent to the scaffold, and
wherein a distal portion of the inflatable balloon defines a tapered transition adjacent to a distal end of the scaffold and a proximal portion of the inflatable balloon defines a tapered transition adjacent to the proximal end of the scaffold.

14. A scaffold delivery apparatus, comprising:
a catheter having an inflatable balloon thereupon;
an expandable scaffold secured upon the inflatable balloon;
a distal spacer positioned beneath or within the inflatable balloon at a location distal to the scaffold,
wherein the distal spacer is tapered to widen from a distal end to a proximal end adjacent to the scaffold, and
wherein the scaffold defines at least a distal tapered edge.

15. A scaffold delivery apparatus, comprising:
a catheter having an inflatable balloon thereupon;
an expandable scaffold secured upon the inflatable balloon;
wherein a distal portion of the inflatable balloon defines a tapered transition adjacent to a distal end of the scaffold and a proximal portion of the inflatable balloon defines a tapered transition adjacent to the proximal end of the scaffold, and
wherein the scaffold defines at least a distal tapered edge.

16. A scaffold delivery apparatus, comprising:
a catheter having an inflatable balloon thereupon;
an expandable scaffold secured upon the inflatable balloon;
a distal spacer positioned beneath or within the inflatable balloon at a location distal to the scaffold,
wherein the distal spacer is tapered to widen from a distal end to a proximal end adjacent to the scaffold,
wherein a distal portion of the inflatable balloon defines a tapered transition adjacent to a distal end of the scaffold and a proximal portion of the inflatable balloon defines a tapered transition adjacent to the proximal end of the scaffold, and
wherein the scaffold defines at least a distal tapered edge.

17. A method of delivery a scaffold, comprising:
providing a catheter having an inflatable balloon thereupon, an expandable scaffold secured upon the inflatable balloon, and a distal spacer positioned beneath or within the inflatable balloon at a location distal to the scaffold, wherein the distal spacer is tapered to widen from a distal end to a proximal end adjacent to the scaffold; and,
inflating the balloon such that the scaffold is expanded radially beyond the distal spacer.
